(19) United States
(12) Patent Application Publication
(10) Pub. No.: US 2024/0218008 A1
(43) Pub. Date: Jul. 4, 2024

(54) SYNTHESIS OF MOLNUPIRAVIR BY GREEN CHEMISTRY

(71) Applicant: AMI LIFESCIENCES PRIVATE LIMITED, Vadodara (IN)

(72) Inventors: Kalpesh Ravajibhai Patel, Vadodara (IN); Virendra Haridas Thakrar, Vadodara (IN); Tushar Bharatkumar Mehta, Vadodara (IN); Atul Gajanan Wagh, Vadodara (IN); Nitin Vasantbhai Prajapati, Vadodara (IN); Nirav Bavanjibhai Sutariya, Vadodara (IN); Jigneshkumar Ashokbhai Patel, Vadodara (IN); Satish Bhausaheb Jadhav, Vadodara (IN)

(73) Assignee: AMI LIFESCIENCES PRIVATE LIMITED, Vadodara (IN)

(21) Appl. No.: 17/918,407

(22) PCT Filed: Feb. 21, 2022
(86) PCT No.: PCT/IB2022/051502

§ 371 Date: Mar. 23, 2023

(30) Foreign Application Priority Data
Sep. 18, 2021 (IN) 202121042313

(51) Int. Cl. C07H 19/067; C07H 1/00

(52) U.S. Cl. CPC C07H 19/067; C07H 1/00

(57) ABSTRACT

The present invention provides improved process for preparation of Molnupiravir compound of formula (I)

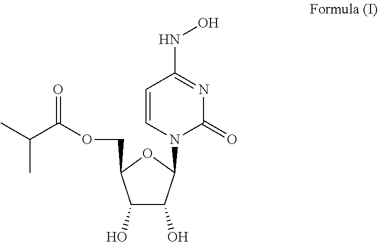 [SMILES: CC(C)C(=O)OC[C@H]1O[C@@H](n2ccc(NO)nc2=O)[C@H](O)[C@@H]1O]

having high purity and yield by avoiding excess use of organic solvent and tedious column chromatography purification. further present invention provides improved process for preparation of compound of formula (V)

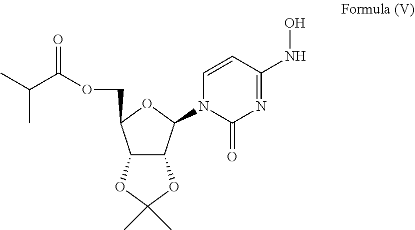 [SMILES: CC(C)C(=O)OC[C@H]1O[C@@H](n2ccc(NO)nc2=O)[C@@H]2OC(C)(C)O[C@@H]21]

in single step reaction having high yield and high purity.

### FIELD OF THE INVENTION

The present invention relates to an improved process for preparation of Molnupiravir (MK-4482, earlier known as EIDD-2801). More particularly, the present invention relates to the improved process parameter such as reaction condition, reaction solvent, isolation solvents, time and avoiding tedious column Chromatography techniques to prepare Molnupiravir which is industrially advantageous, eco-friendly and economically significant.

### BACKGROUND OF THE INVENTION

Molnupiravir is chemically known as [(2R,3S,4R,5R)-3,4-dihydroxy-5-[4-(hydroxyamino)-2-oxopyrimidin-1-yl]oxolan-2-yl]methyl 2-methylpropanoate, having the structure compound of formula (I)

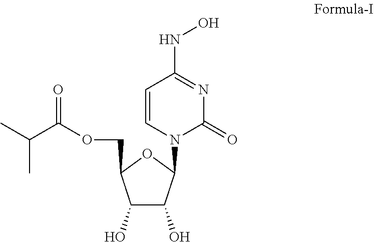 [SMILES: CC(C)C(=O)OC[C@H]1O[C@@H](n2ccc(NO)nc2=O)[C@H](O)[C@@H]1O]

Mohupiravir was discovered at Emory University and is undergoing clinical development in partnership with Ridgeback Biopharmnaceuticals and Merck & Co. Molnupiravir is in development by Merck after licensing from Ridgeback Biopharmaceuticals as an orally dosed antiviral for the treatment of COVID-19.

PCT publication WO 2019113462A1 (herein after WO '462) first discloses process for preparation of Molnupiravir comprising reacting uridine with acetone to obtain protected compound of formula (A) without purification, further it reacts with 2-methylpropanoyl 2-methylpropanoate in presence of base to obtain compound of formula (B), further it reacts with 1,2,4-triazole to obtain compound of formula (C). Further compound of formula (C) reacts with hydroxylamine to obtain compound of formula (D), further deprotecting compound of formula (D) using acid to obtain Molnupiravir as crude product, further purification by IPA/MTBE to obtain pure Molnupiravir compound of formula (I).

The schematic representation of Molnupiravir process as disclosed in WO '462 is given below in scheme-I.

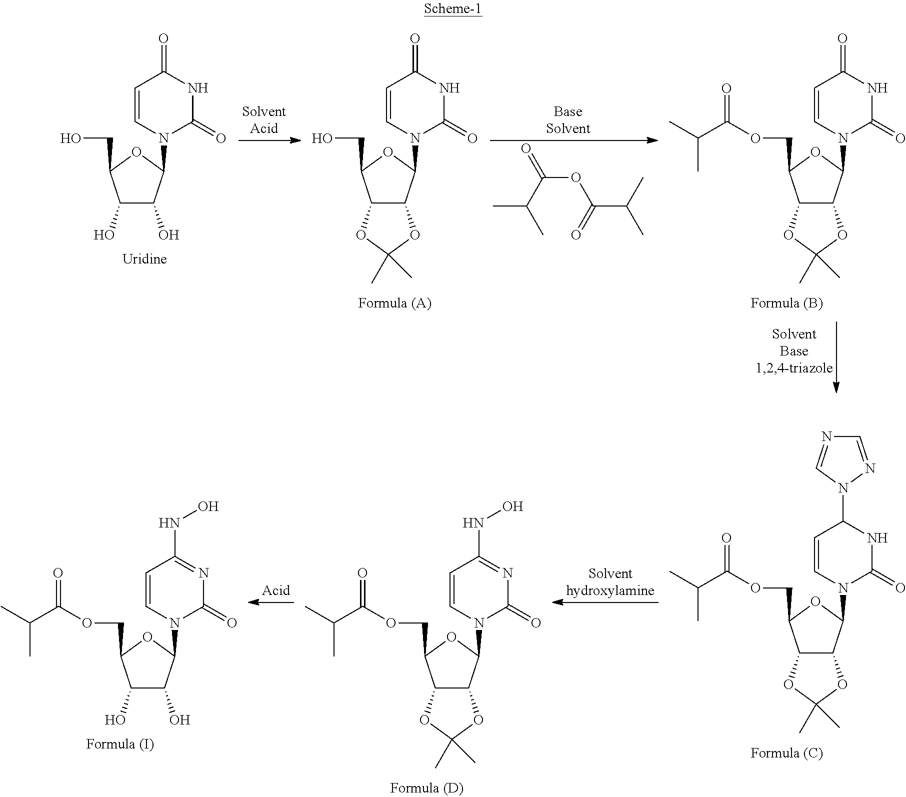

The process as disclosed in WO '462 has several disadvantages such as using uridine expensive key raw material, further during process many impurities are generated which is carried forward in the final Molnupiravir API. The present invention using cytidine as key raw material which is less expensive as compared to uridine. The present invention overcomes all the disadvantages of WO '462 process.

Synlett, Volume 32, Issue 3, Pages 326-328, Journal 2021 discloses the process for preparation of Molnupiravir comprising reacting cytidine compound of formula (I) with 2,2-dimethoxypropane followed by acetone addition in presence of H₂SO₄ without any purification to obtain sulphate salts of compound of formula (III), further compound of formula (III) is reacting with isobutyric anhydride followed by addition of DBU and DMAP, followed by column chromatography purification (Methanol & CHCl₃) to obtain compound of formula (IV), further it reacting with hydroxylamine sulfate followed by addition of IPA solvent and isolating in toluene solvent to obtain compound of formula (V). Further compound of formula (V) is reacted with formic acid followed by isolation in MTBE/IPA, further purified by column chromatography in Methanol & CHCl₃ to obtain Molnupiravir compound of formula I having 60% yield.

The schematic representation of Molnupiravir process as disclosed in Synlett, Volume 32, Issue 3, Pages 326-328, Journal 2021 is given below in scheme-II.

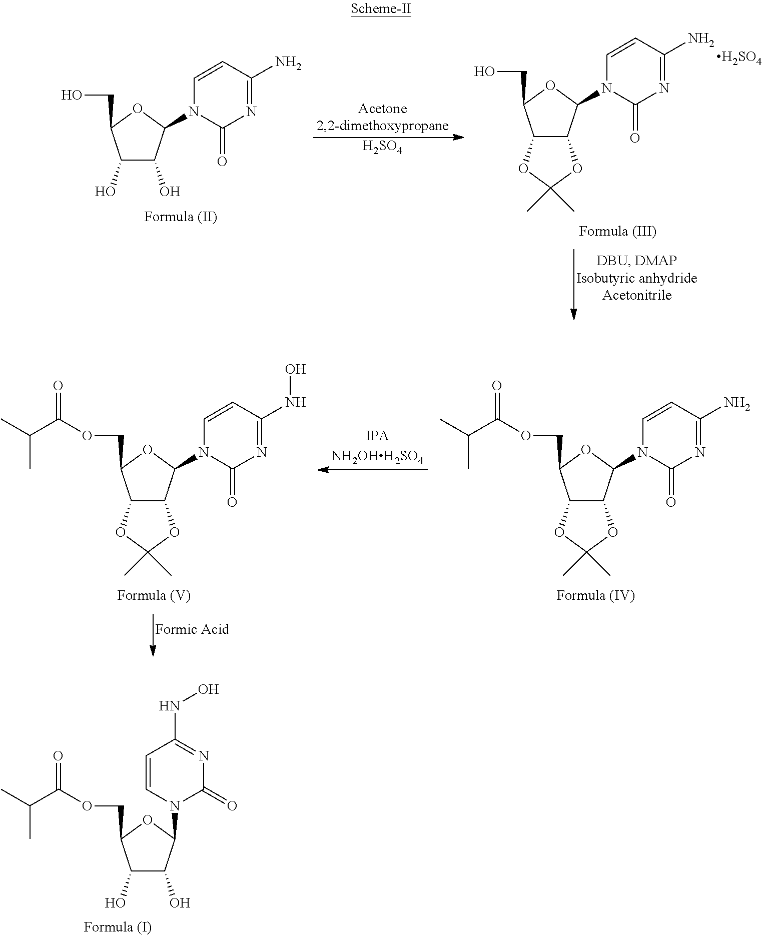

The major disadvantage of process as disclosed in Synlett, Volume 32, Issue 3, Pages 326-328, Journal 2021 is that we observed release of carbon monoxide during the reaction with formic acid used in deprotection of compound of formula (V). However, it is also in the literature that formic acid is a source of carbon monoxide and mere storing it for a longer time result in the liberation of carbon monoxide. Another one disadvantage process as disclosed in Synlett, Volume 32, Issue 3, Pages 326-328, Journal 2021 is purification of compound formula (IV) & compound of formula (I) by column chromatography and using large volume of IPA reaction solvent during formation of compound of formula (V) which is not suitable for large scale industrial production of Molnupiravir compound of formula (I).

All the prior arts discussed above suffer from many disadvantages like tedious and cumbersome work up procedures, high temperature condition, longer reaction times, use of excess reagent and solvents and column chromatographic purification which affect the overall yield as well as the quality of the final product.

Therefore, there is a need for improved process for the preparation of Molnupiravir having high purity and high yield which overcome the drawbacks of the prior arts process. The present inventors surprisingly found an efficient process for the preparation of Molnupiravir which offer advantage over the prior art processes in term of reaction time, high yield, high purity, less use of reaction solvent, avoiding column chromatography purification, less effluents and simple scalable procedure suitable for large scale industrial production.

### OBJECT OF THE INVENTION

The principal object of the present invention is to provide process for preparation of Molnupiravir compound of formula (I) by avoiding excess use of organic solvent in order to prepare Molnupiravir compound of formula (I) having high yield and high purity, therefore present invention towards green chemistry.
a) Isolation of the intermediate compound of formula (IV) is avoided by present invention, which was reported in prior art Synlett, Volume 32, Issue 3, Pages 326-328, Journal 2021 by column chromatography, the same is considered as in-situ compound of formula (IV) which is used for preparation of Molnupiravir compound of formula (I).
b) Conversion of in-situ compound of formula (IV) to compound of formula (V) is done by using water as reaction solvent, whereas prior art process uses large volume of alcohol solvent.

### SUMMARY OF INVENTION

First aspect of the present invention is relates to a process for the preparation of Molnupiravir compound of formula (I)

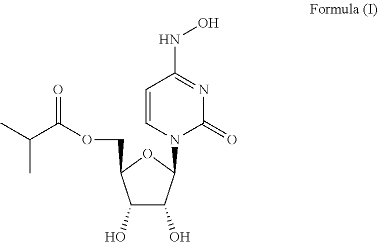 [SMILES: CC(C)C(=O)OC[C@H]1O[C@@H](n2ccc(NO)nc2=O)[C@H](O)[C@@H]1O]

comprising steps of:
a) reacting compound of formula (III) in solvent

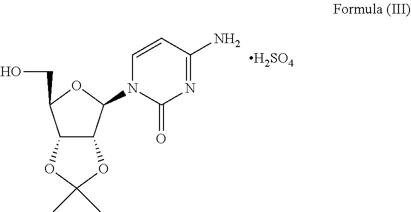

with isobutyric anhydride in presence of base and catalyst under suitable condition to obtain in-situ compound of formula (IV);

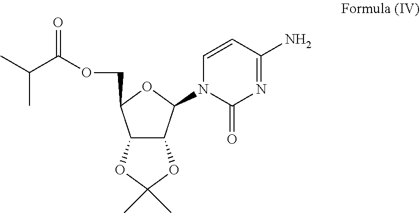 [SMILES: CC(C)C(=O)OC[C@H]1O[C@@H](n2ccc(N)nc2=O)[C@@H]2OC(C)(C)O[C@@H]21]

b) reacting compound of formula (IV) with hydroxyl amine sulfate in water solvent to obtain compound of formula (V); and

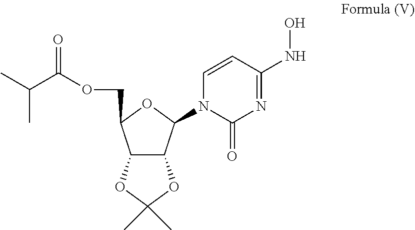 [SMILES: CC(C)C(=O)OC[C@H]1O[C@@H](n2ccc(NO)nc2=O)[C@@H]2OC(C)(C)O[C@@H]21]

c) converting compound of formula (V) to Molnupiravir compound of formula (I).

Second aspect of the present invention is relates to a process for preparation of compound of formula (V)

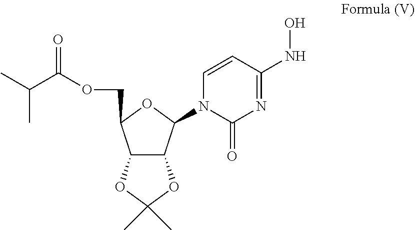 [SMILES: CC(C)C(=O)OC[C@H]1O[C@@H](n2ccc(NO)nc2=O)[C@@H]2OC(C)(C)O[C@@H]21]

comprising steps of:
a) reacting compound of formula (III) in solvent

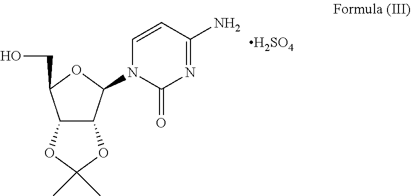

with isobutyric anhydride in presence of base and catalyst under suitable condition to obtain in-situ compound of formula (IV); and

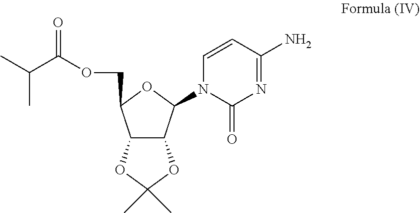 [SMILES: CC(C)C(=O)OC[C@H]1O[C@@H](n2ccc(N)nc2=O)[C@@H]2OC(C)(C)O[C@@H]21]

b) reacting compound of formula (IV) with hydroxyl amine sulfate in water solvent to obtain compound of formula (V);

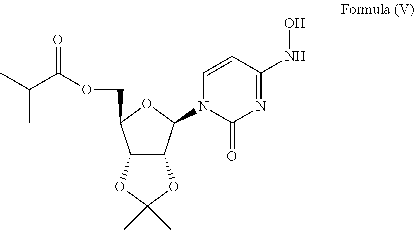 [SMILES: CC(C)C(=O)OC[C@H]1O[C@@H](n2ccc(NO)nc2=O)[C@@H]2OC(C)(C)O[C@@H]21]

Third aspect of the present invention is relates to improved isolation process for preparation of compound of formula (V)

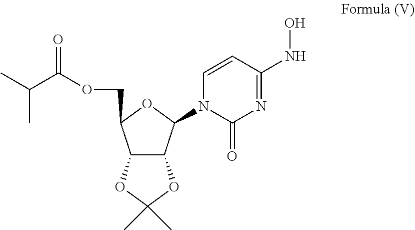 [SMILES: CC(C)C(=O)OC[C@H]1O[C@@H](n2ccc(NO)nc2=O)[C@@H]2OC(C)(C)O[C@@H]21]

comprising steps of:
a) reacting compound of formula (III) in solvent

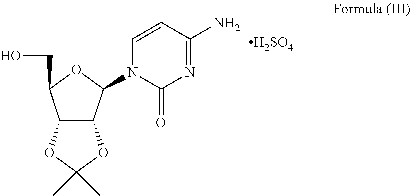

with isobutyric anhydride in presence of base and catalyst under suitable condition to obtain in-situ compound of formula (IV);

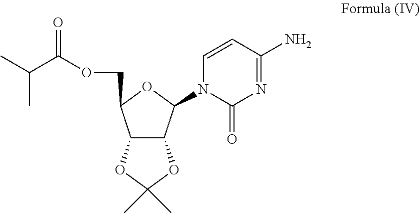 [SMILES: CC(C)C(=O)OC[C@H]1O[C@@H](n2ccc(N)nc2=O)[C@@H]2OC(C)(C)O[C@@H]21]

b) reacting compound of formula (IV) with hydroxyl amine sulfate in water solvent; and
c) isolating compound of formula (V) in alcohol solvent.

Fourth aspect of the present invention is relates to process for preparation of Molnupiravir compound of formula (I) comprises:

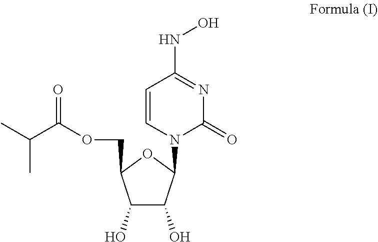 [SMILES: CC(C)C(=O)OC[C@H]1O[C@@H](n2ccc(NO)nc2=O)[C@H](O)[C@@H]1O]

by using water as solvent for reacting compound of formula (IV)

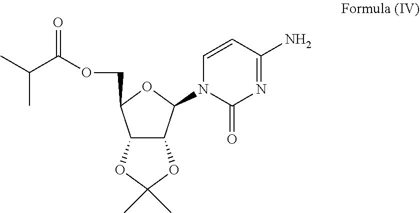 [SMILES: CC(C)C(=O)OC[C@H]1O[C@@H](n2ccc(N)nc2=O)[C@@H]2OC(C)(C)O[C@@H]21]

with hydroxyl amine sulfate at suitable reaction conditions to obtain compound of formula (V); and

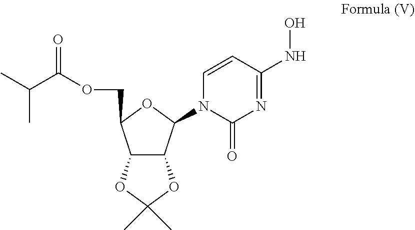 [SMILES: CC(C)C(=O)OC[C@H]1O[C@@H](n2ccc(NO)nc2=O)[C@@H]2OC(C)(C)O[C@@H]21]

converting compound of formula (V) to Molnupiravir formula (I).

### Definition

All percentages and ratios used herein are by weight of the total composition and all measurements made are at 25° C. and normal pressure unless otherwise designated.

All temperatures used herein are in degrees Celsius unless specified otherwise.

All ranges recited herein include the endpoints, including those that recite a range “between” two values.

As used herein, “comprising” means the elements recited, or their equivalents in structure or function, plus any other element or elements that may or may not be recited.

The terms “having” and “including” are also to be construed as open ended unless the context suggests otherwise.

### DETAIL DESCRIPTION OF INVENTION

While the following specification concludes with claims particularly pointing out and distinctly claiming the invention, it is anticipated that the invention can be more readily understood through reading the following detailed description and also by studying the included examples

The best methods and materials of performing the present invention are described here.

In an attempt to develop an improved process for the preparation of Molnupiravir and to overcome the disadvantages of prior art, the present inventors have developed a process which results in high purity and good yield of Molnupiravir.

The process for preparation of Molnupiravir as per the present invention can be summarized in the following schematic representation.

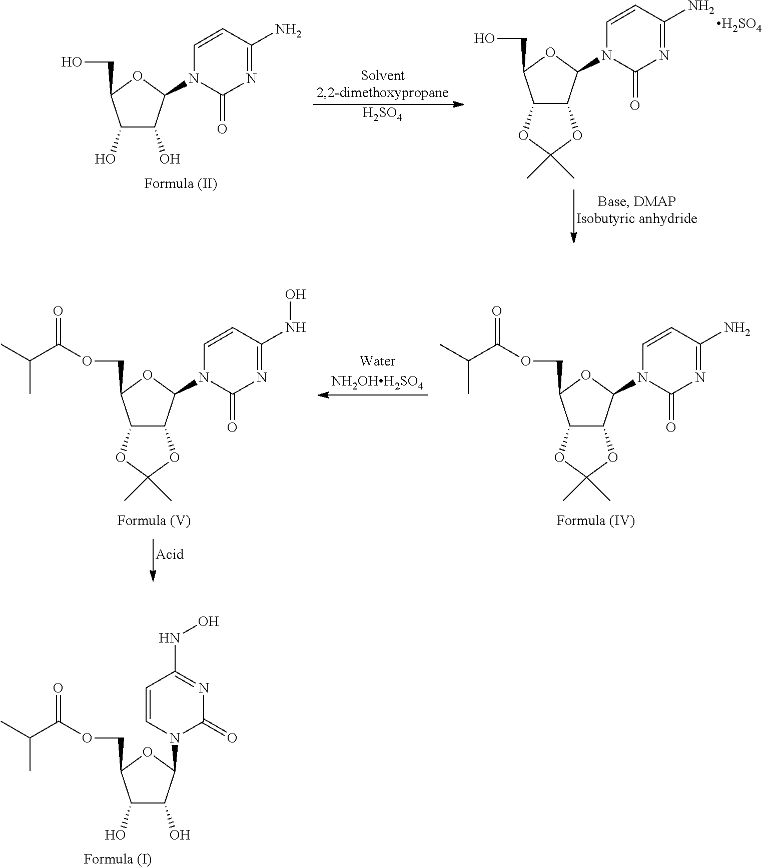

The starting material compound of formula (III) of the present invention is commercially available, or can be prepared by known prior art process or can be prepared by process as disclosed in Synlett, Volume 32, Issue 3, Pages 326-328, Journal 2021 herein incorporated reference purpose only.

In the first embodiment present invention provide a process for the preparation of Molnupiravir compound of formula (I)

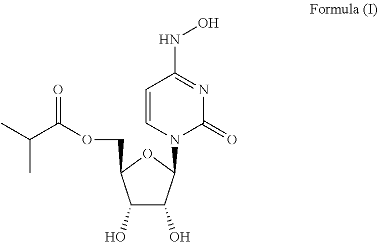 [SMILES: CC(C)C(=O)OC[C@H]1O[C@@H](n2ccc(NO)nc2=O)[C@H](O)[C@@H]1O]

comprising steps of:
a) reacting compound of formula (III) in solvent

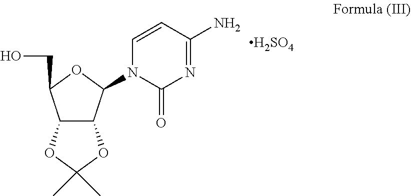

with isobutyric anhydride in presence of base and catalyst under suitable condition to obtain in-situ compound of formula (IV);

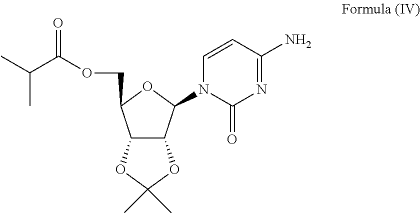 [SMILES: CC(C)C(=O)OC[C@H]1O[C@@H](n2ccc(N)nc2=O)[C@@H]2OC(C)(C)O[C@@H]21]

b) reacting compound of formula (IV) with hydroxyl amine sulfate in water solvent to obtain compound of formula (V); and

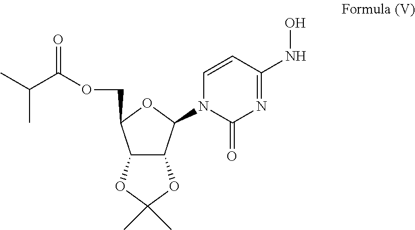 [SMILES: CC(C)C(=O)OC[C@H]1O[C@@H](n2ccc(NO)nc2=O)[C@@H]2OC(C)(C)O[C@@H]21]

c) converting compound of formula (V) to Molnupiravir compound of formula (I).

In the embodiment of step, a) solvent selected from polar aprotic solvent such as tetrahydrofuran (THF), dimethylsulfoxide (DMSO), dimethylformamide (DMF), acetonitrile, dichloromethane (MDC) and mixture thereof; more preferably polar aprotic solvent is acetonitrile.

In the embodiment of step, a) base selected from organic bases such as triethylamine, diisopropylethylamine, morpholine, N-methyl morpholine, DABCO and like the; more preferably organic base is triethylamine.

In the embodiment of step, a) catalyst is 4-Dimethylaminopyridine (DMAP).

In the embodiment of step, a) is suitably carried out at temperature in the range of 30-40° C.; more preferably reaction is carried out at temperature in the range of 25-35° C. and step, a) is suitably carried at reaction time 1-4 hours; more preferably reaction is carried out at 1-2 hours.

In the embodiment of step, a) compound of formula (IV) is not isolating, which is used in the next step without any further purification.

In the second embodiment of the present invention is to provide process for preparation of compound of formula (V)

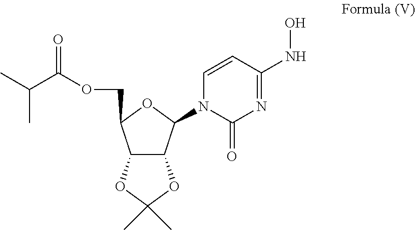 [SMILES: CC(C)C(=O)OC[C@H]1O[C@@H](n2ccc(NO)nc2=O)[C@@H]2OC(C)(C)O[C@@H]21]

comprising steps of:
a) reacting compound of formula (III) in solvent

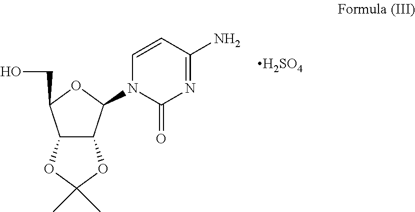

with isobutyric anhydride in presence of base and catalyst under suitable condition to obtain in-situ compound of formula (IV); and

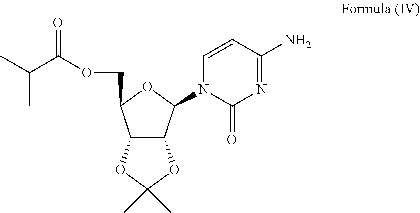 [SMILES: CC(C)C(=O)OC[C@H]1O[C@@H](n2ccc(N)nc2=O)[C@@H]2OC(C)(C)O[C@@H]21]

b) reacting compound of formula (IV) with hydroxyl amine sulfate in water solvent to obtain compound of formula (V);

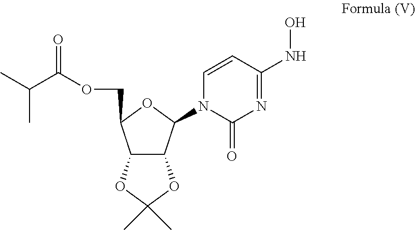 [SMILES: CC(C)C(=O)OC[C@H]1O[C@@H](n2ccc(NO)nc2=O)[C@@H]2OC(C)(C)O[C@@H]21]

In the embodiment of step, a) solvent polar aprotic solvent such as tetrahydrofuran (THF), dimethylsulfoxide (DMSO), dimethylformamide (DMF), acetonitrile, dichloromethane (MDC) and mixture thereof; more preferably polar aprotic solvent is acetonitrile.

In the embodiment of step, a) base selected from organic bases such as triethylamine, diisopropylethylamine, morpholine, N-methyl morpholine, DABCO and like the; more preferably organic base is triethylamine.

In the embodiment of step, a) catalyst is 4-Dimethylaminopyridine (DMAP).

In the embodiment of step, a) is suitably carried out at temperature in the range of 30-40° C.; more preferably reaction is carried out at temperature in the range of 25-35° C. and step, b) is suitably carried at reaction time 1-4 hours; more preferably reaction is carried out at 1-2 hours.

In the embodiment of step, a) compound of formula (IV) is not isolating, which is used in the next step without any further purification.

In the third embodiment of the present invention is to provide improved isolation process for preparation of compound of formula (V)

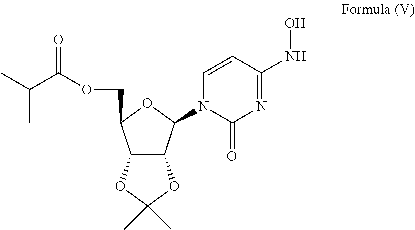 [SMILES: CC(C)C(=O)OC[C@H]1O[C@@H](n2ccc(NO)nc2=O)[C@@H]2OC(C)(C)O[C@@H]21]

comprising steps of:
a) reacting compound of formula (III) in solvent

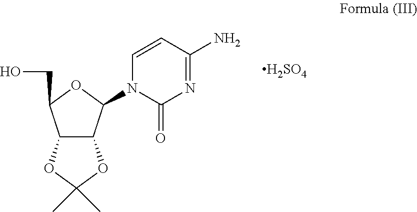

with isobutyric anhydride in presence of base and catalyst under suitable condition to obtain in-situ compound of formula (IV);

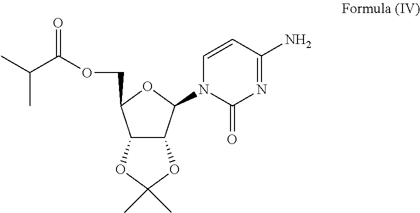 [SMILES: CC(C)C(=O)OC[C@H]1O[C@@H](n2ccc(N)nc2=O)[C@@H]2OC(C)(C)O[C@@H]21]

b) reacting compound of formula (IV) with hydroxyl amine sulfate in water solvent; and
c) isolating compound of formula (V) in alcohol solvent.

In the embodiment of step, a) solvent is selected from polar aprotic solvent such as tetrahydrofuran (THF), dimethylsulfoxide (DMSO), dimethylformamide (DMF), acetonitrile, dichloromethane (MDC) and mixture thereof; more preferably polar aprotic solvent is acetonitrile.

In the embodiment of step, a) base selected from organic bases such as triethylamine, diisopropylethylamine, morpholine, N-methyl morpholine, DABCO and like the; more preferably organic base is triethylamine.

In the embodiment of step, a) compound of formula (IV) is not isolating, which is used in the next step without any further purification.

In the embodiment of step, a) catalyst is 4-Dimethylaminopyridine (DMAP).

In the embodiment of step, b) is suitably carried out temperature in the range of 30-70° C.; more preferably reaction is carried out at temperature in the range of 60-65° C. and step, b) is suitably carried at reaction time 1-12 hours; more preferably reaction is carried out at 10-12 hours.

In the embodiment of step, c) compound of formula (V) is isolating in alcohol solvent selected from methanol, ethanol, 2-Methyl-1-butanol, 2-Methyl-1-pentanol, 3-Methyl-2-butanol, Isobutanol, Isopropyl alcohol (IPA), 1-Propanol, 2-Pentanol and mixture thereof; preferably alcohol is Isopropyl alcohol (IPA).

In the embodiment of step, c) the compound of formula (V) is isolating directly by filtration in the reaction mass.

In the fourth embodiment of the present invention provide process for preparation of Molnupiravir compound of formula (I) comprises:

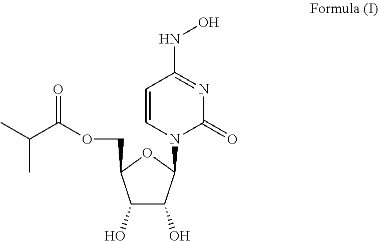 [SMILES: CC(C)C(=O)OC[C@H]1O[C@@H](n2ccc(NO)nc2=O)[C@H](O)[C@@H]1O]

by using water as solvent for reacting compound of formula (IV)

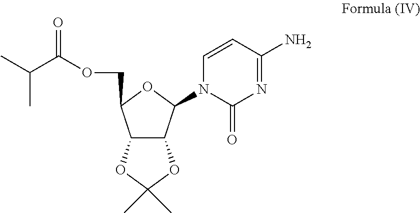 [SMILES: CC(C)C(=O)OC[C@H]1O[C@@H](n2ccc(N)nc2=O)[C@@H]2OC(C)(C)O[C@@H]21]

with hydroxyl amine sulfate at suitable reaction conditions to obtain compound of formula (V); and

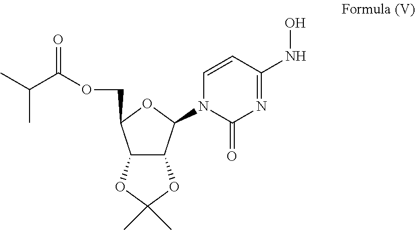 [SMILES: CC(C)C(=O)OC[C@H]1O[C@@H](n2ccc(NO)nc2=O)[C@@H]2OC(C)(C)O[C@@H]21]

converting compound of formula (V) to Molnupiravir formula (I).

In the fourth embodiment converting of compound of formula (IV) to compound of formula (V) in water as solvent reaction is carried out at temperature in the range of 20-70° C.; more preferably reaction is to be carried out at temperature 60-65° C.

In the fourth embodiment converting of compound of formula (IV) to compound of formula (V) in water as solvent reaction is carried out at time in the range of 1-14 hours; more preferably reaction is to be carried out at time 8-12 hours.

In the fourth embodiment converting of compound of formula (V) to Molnupiravir compound of formula (I) reaction is carried out at temperature in the range of 20-40° C.; more preferably reaction is to be carried out at temperature 25-35° C.

In the fourth embodiment converting of compound of formula (V) to Molnupiravir compound of formula (I) reaction is carried out at time in the range of 1-10 hours; more preferably reaction is to be carried out at time 1-8 hours.

### EXAMPLE

The following examples are illustrative of some of the embodiments of the present, invention described herein. These examples should not be considered to limit the spirit or scope of the invention in any way.

### Example 01: Preparation of Compound of Formula (III)

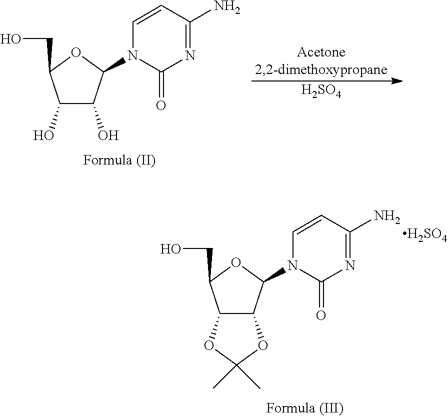

Under nitrogen charged 1300 ml acetone, 100 gm cytidine compound of formula (II), 217 gm 2-Dimethoxy propane at 25-35° C. in RBF. Added 44 gm sulfuric acid at 25-35° C., stirred for 4 hrs at 25-35° C. Checked HPLC Analysis cytidine should be not more than 0.5%. Filtered the reaction mass at 25-35° C. Washed it with 200 ml acetone at 25-35° C. Charged 600 ml acetone and wet cake in RBF. Stirred for 60 min at 25-35° C. Filtered at 25-35° C. and washed with 200 ml acetone. Unloaded the wet cake and dried in VTD at 55-60° C. to obtained 150 gm compound of formula (III).
HPLC Purity: 99.2%.
Yield: 95%
M.P: 250° C.
m/z: 283.94 (M-H2SO4)

### Example 02: Preparation of Compound of Formula (V)

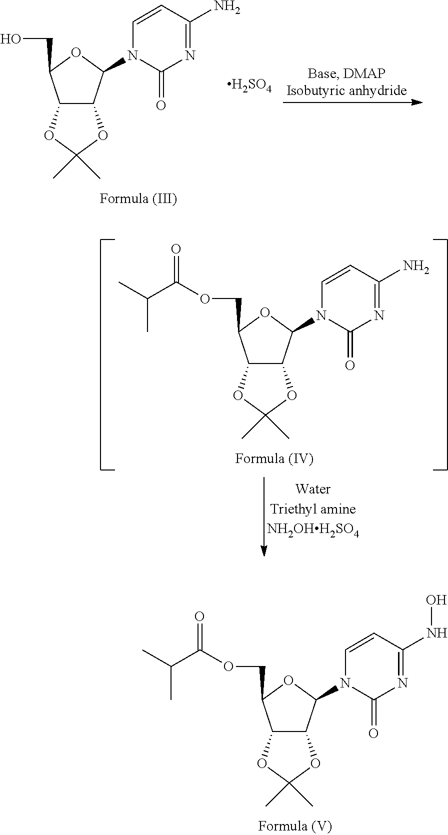

Under Nitrogen, charged 400 ml acetonitrile, 100 gm Compound of formula (III), 6.5 gm 4-Dimethyl amino pyridine at 25-35° C. in RBF. Added 56 gm Triethyl amine and 45 gm isobutyric anhydride at 0-5° C., stirred for 1-2 hrs and checked HPLC Analysis. Compound of formula (II) should be not more than 0.5%. Distilled out acetonitrile under vacuum below 50° C. to obtained residue of intermediate compound of formula (IV).

Charged 500 ml water, 8 gm triethyl amine, 114 gm Hydroxyl amine Sulfate at 25-35° C. Maintained temperature to 60-65° C. for 12 hrs and checked HPLC intermediate compound of formula (IV) should be NMT 1.0%. Filtered the reaction mass at 20-25° C. after 1 hr stirred washed it with 100 ml water, unloaded the wet cake. Charged wet cake in 425 ml Isopropyl alcohol (IPA). Distilled out isopropyl alcohol to get thick slurry below 50° C. under vacuum. Charged 125 ml isopropyl alcohol (IPA); Filtered the reaction mass at 0-5° C. after 1 hr stirred. Unloaded the wet cake and dried in VTD at 40-50° C. to obtained compound of formula (V) 71 gm.
Yield: 73.37%
Purity by HPLC: 99.2%.
M.P: 158-160° C.
m/z: 391.75 (M+Na)

### Example 03: Preparation of Compound of Formula (I)

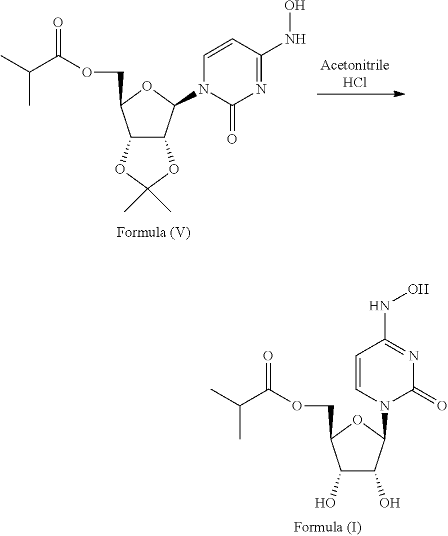 [SMILES: CC(C)C(=O)OC[C@H]1O[C@@H](n2ccc(NO)nc2=O)[C@@H]2OC(C)(C)O[C@@H]21 ; CC(C)C(=O)OC[C@H]1O[C@@H](n2ccc(NO)nc2=O)[C@H](O)[C@@H]1O]

Charged 1000 ml Acetonitrile, 100 gm compound of formula (V) into the RBF at 25-35° C. Added 29.6 gm hydrochloric acid at 25-30° C. Maintained the reaction mass for 8 hrs. at 25-30° C. Checked UPLC compound of formula (V) should be NMT 10.0% and compound of formula (I) NLT 83%. Added 19 gm Sodium carbonate, 40 ml water at 25-35° C. Distilled out acetonitrile at NMT 50° C. till to obtained residue. Stripped out 30 ml ethyl acetate. Charged 300 ml water, 40 gm sodium sulphate followed by extraction with 4×500 ml ethyl acetate at 35-40° C. Separated out organic layer and washed it with 2×100 ml saturated brine solution at 35-40° C. Distilled out organic layer NMT 50° C. under vacuum to obtained residue, followed by addition of 500 ml ethyl acetate. Maintained for 1 hr at 50-55° C. Cooled and filtered it after maintaining for 1 hr at 0-5° C., washed it with 70 ml ethyl acetate. unloaded the wet cake and dried in VTD at 40-50° C. to obtained 70 gm compound of formula (I).
HPLC Purity: 99.6%.
Yield: 78%

### Example 04: Purification of Compound of Formula (I)

Charged 700 ml isopropyl alcohol, 100 gm compound of formula (I) and 2 gm carbon 25-35° C., stirred it for 30 min at 70-75° C., filtered it on hyflo and washed it with hot 100 ml isopropyl alcohol (IPA). Followed by distillation of isopropyl alcohol (IPA) up to reaction mass 250 ml at NMT 60° C. under vacuum. Added 300 ml methyl tertiary butyl ether (MTBE) at 50-55° C. Cooled and filtered it after maintaining for 1 hr at 0-5° C., washed it with 50 ml methyl tertiary butyl ether (MTBE). unloaded the wet cake and dried in VTD at 40-50° C. to obtained 90 gm Molnupiravir compound of formula (I).
HPLC Purity: 99.75%
Yield: 90%
M.P: 162-164° C.
m/z: 328.07 (M-1)
¹H NMR (400 MHZ, CD₃OD): δ 6.82 (d, J=8.4 Hz, 1H), 5.73 (d, J=4.8 Hz, 1H), 5.54 (d, J=8.0 Hz, 1H), 4.20 (d, J=3.6 Hz, 2H), 4.06 (t, J=4.9 Hz, 3H), 2.55 (multiplet, J=7.2 Hz, 1H), 1.13 (d, J=6.8 Hz, 6H);
¹³C NMR (100 MHZ, CD₃OD): δ 178.35 151.60, 146.23, 131.82, 99.65, 90.54, 82.61, 74.45, 71.54, 64.99, 35.19, 19.43, 19.39

1. A process for the preparation of Molnupiravir compound of formula (I) comprises: 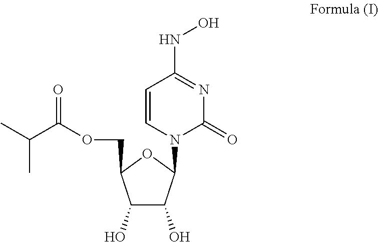 [SMILES: CC(C)C(=O)OC[C@H]1O[C@@H](n2ccc(NO)nc2=O)[C@H](O)[C@@H]1O]
by using water as solvent for reacting compound of formula (IV) 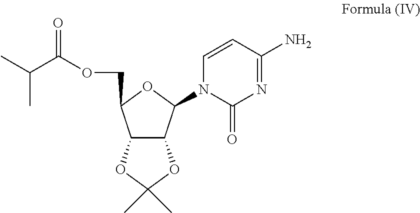 [SMILES: CC(C)C(=O)OC[C@H]1O[C@@H](n2ccc(N)nc2=O)[C@@H]2OC(C)(C)O[C@@H]21]
with hydroxyl amine sulfate at suitable reaction conditions to obtain compound of formula (V); and 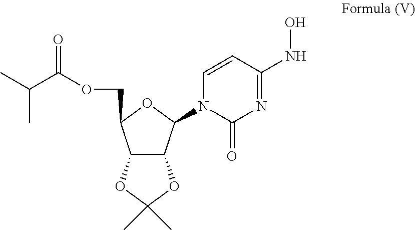 [SMILES: CC(C)C(=O)OC[C@H]1O[C@@H](n2ccc(NO)nc2=O)[C@@H]2OC(C)(C)O[C@@H]21]
converting compound of formula (V) to Molnupiravir formula (I).

2. The process as claimed in claim 1; wherein converting of compound of formula (IV) to compound of formula (V) in water as solvent is carried out at temperature in the range of 20-70° C.

3. The process as claimed in claim 1; wherein converting of compound of formula (IV) to compound of formula (V) in water as solvent is carried out at time in the range of 1-14 hours; more preferably reaction is to be carried out at time of 8-12 hours.

4. The process as claimed in claim 1; wherein converting compound of formula (IV) to compound of formula (V) with purity 99.2%.

5. The process as claimed in claim 1; wherein converting of compound of formula (V) to Molnupiravir compound of formula (I) is carried out at temperature in the range of 20-40° C.

6. The process as claimed in claim 1; wherein converting of compound of formula (V) to Molnupiravir compound of formula (I) is carried out at time in the range of 1-10 hours.

7. The process as claimed in claim 2; wherein the reaction is to be carried out at temperature of 60-65° C.

8. The process as claimed in claim 5, wherein the reaction is to be carried out at temperature of 25-35° C.

8. The process as claimed in claim 6, wherein the reaction is to be carried out at time of 1-8 hours.
